(19) United States
(12) Patent Application Publication
(10) Pub. No.: US 2022/0299508 A1
(43) Pub. Date: Sep. 22, 2022

(54) RAPID DIAGNOSTIC TEST COMPONENT

(71) Applicant: Detect, Inc., Guilford, CT (US)

(72) Inventors: Jonathan M. Rothberg, Miami Beach, FL (US); Isaac Bean, Colorado Springs, CO (US); Todd Roswech, Ivoryton, CT (US)

(73) Assignee: Detect, Inc., Guilford, CT (US)

(21) Appl. No.: 17/694,511

(22) Filed: Mar. 14, 2022

Related U.S. Application Data
(60) Provisional application No. 63/161,886, filed on Mar. 16, 2021.

(51) Int. Cl. G01N 33/543; B01L 3/00

(52) U.S. Cl. CPC G01N 33/54388; B01L 3/5023; B01L 3/523; B01L 2300/0672; B01L 2300/069; B01L 2300/0835; B01L 2400/0683

(57) ABSTRACT

Provided herein, in some embodiments, are rapid diagnostic tests to detect one or more target nucleic acid sequences (e.g., a nucleic acid sequence of one or more pathogens). In some embodiments, the pathogens are viral, bacterial, fungal, parasitic, or protozoan pathogens, such as SARS-CoV-2 or an influenza virus. Further embodiments provide methods of detecting genetic abnormalities. Diagnostic tests comprising a sample-collecting component, one or more reagents (e.g., lysis reagents, nucleic acid amplification reagents), and a detection component (e.g., a component comprising a lateral flow assay strip) are provided.

### RELATED APPLICATIONS

This application claims the benefit of priority under 35 U.S.C. § 119(e) of U.S. Provisional App. Ser. No. 63/161,886, filed Mar. 16, 2021, the disclosure of which is herein incorporated by reference in its entirety.

### FIELD

The present invention generally relates to diagnostic devices, systems, and methods for detecting the presence of a target nucleic acid sequence.

### BACKGROUND

The ability to rapidly diagnose diseases—particularly highly infectious diseases—is critical to preserving human health. As one example, the high level of contagiousness, the high mortality rate, and the lack of a treatment or vaccine for the coronavirus disease 2019 (COVID-19) have resulted in a pandemic that has already infected millions and killed hundreds of thousands of people. The existence of rapid, accurate COVID-19 diagnostic tests could allow infected individuals to be quickly identified and isolated, which could assist with containment of the disease. In the absence of such diagnostic tests, COVID-19 may continue to spread unchecked throughout communities.

### SUMMARY

Provided herein are a number of diagnostic tests useful for detecting target nucleic acid sequences. The tests, as described herein, are able to be performed in a point-of-care (POC) setting or home setting without specialized equipment. Therefore, in some aspects, the disclosure provides a diagnostic test including an absorbent pad containing a first solution as well as a lateral flow assay strip. The absorbent pad may be in fluid communication with the lateral flow assay strip, and may be configured to receive a sample, so that the sample and the first solution (e.g., a diluent) mix in the absorbent pad before flowing to the lateral flow assay strip.

In some embodiments, a component of a diagnostic test includes an absorbent pad at least partially saturated with a first solution, where the pad is fluidly coupled between a reservoir that receives a sample and a readout element, and where the absorbent pad enables the sample to flow between the reservoir and readout element through the absorbent pad. In some embodiments, the readout element is a lateral flow assay strip.

In some embodiments, a detection component of a diagnostic test includes a lateral flow assay strip, an absorbent pad fluidly connected to the lateral flow assay strip, where the absorbent pad is at least partially saturated with a first solution, and a receptacle configured to receive and fluidly connect to a first reservoir containing a sample, where fluidly connecting the first reservoir to the receptacle allows the sample to flow from the first reservoir to the absorbent pad.

In some embodiments, a diagnostic test kit includes a vial containing a first solution and configured to receive a sample and a detection component. The detection component includes a receptacle configured to receive the vial, an absorbent pad, and a lateral flow assay strip fluidly connected to the absorbent pad.

In some embodiments, a method of performing a diagnostic test includes depositing a sample in a first reservoir, moving the first reservoir into a receptacle of a detection component, and fluidly connecting the first reservoir with the receptacle to allow the sample to flow to an absorbent pad at least partially saturated with a first solution, wherein the absorbent pad is fluidly connected to a lateral flow assay strip.

In some embodiments, a method of performing a diagnostic test includes depositing a sample in a first reservoir, depositing a first solution into a receptacle of a detection component, allowing the first solution to flow to an absorbent pad to at least partially saturate the absorbent pad with the first solution, and fluidly connecting the first reservoir with the receptacle to allow the sample to flow to the absorbent pad at least partially saturated with the first solution, where the absorbent pad is fluidly connected to a lateral flow assay strip.

In some embodiments, a method of manufacturing a diagnostic test includes placing a lateral flow assay strip and an absorbent pad in a housing, wherein the lateral flow assay strip is in fluid communication with the absorbent pad, at least partially filling the absorbent pad with a first solution, and providing a vial for taking a sample from a patient, wherein the vial is configured to fluidly connect to the housing, and wherein fluidly connecting the vial allows the sample to flow to the absorbent pad.

In some embodiments, a method of manufacturing a diagnostic test includes placing a lateral flow assay strip and an absorbent pad in a housing, where the lateral flow assay strip is in fluid communication with the absorbent pad, filling a dropper with a first solution, and providing a vial for taking a sample from a patient, wherein the vial is configured to fluidly connect to the housing, and wherein fluidly connecting the vial allows the sample to flow to the absorbent pad.

It should be appreciated that the foregoing concepts, and additional concepts discussed below, may be arranged in any suitable combination, as the present disclosure is not limited in this respect. Further, other advantages and novel features of the present disclosure will become apparent from the following detailed description of various non-limiting embodiments when considered in conjunction with the accompanying figures.

### BRIEF DESCRIPTION OF DRAWINGS

The accompanying drawings are not intended to be drawn to scale. In the drawings, each identical or nearly identical component that is illustrated in various figures may be represented by a like numeral. For purposes of clarity, not every component may be labeled in every drawing. In the drawings:

FIGS. 1A-1F show, according to some embodiments, a process of performing a diagnostic test for the presence of one or more nucleic acid sequences;

FIGS. 2A-2C show, according to some embodiments, a detection component comprising a “chimney” and an absorbent pad;

FIG. 3 shows, according to some embodiments, a flow chart for a method of performing a diagnostic test;

FIG. 4 shows, according to some embodiments, a flow chart for a method of manufacturing a diagnostic test;

FIGS. 5A-5D show, according to some embodiments, a detection component comprising a “chimney” and an absorbent pad;

FIG. 6 shows, according to some embodiments, a flow chart for a method of performing a diagnostic test;

FIG. 7 shows, according to some embodiments, a flow chart for a method of manufacturing a diagnostic test;

FIG. 8 shows, according to some embodiments, an exploded view of a detection component comprising a “chimney”;

FIGS. 9A-9B show diagnostic kits comprising a sample-collecting component, a reaction tube, a detection component, and a heater, according to some embodiments;

FIG. 10A-10D shows, according to some embodiments, a detection component comprising blisters and an absorbent pad; and

FIG. 11 shows, according to some embodiments, a flow chart for a method of manufacturing a diagnostic test.

### DETAILED DESCRIPTION

Conventional nucleic acid tests for various diseases requires trained medical professional to collect samples and process those samples in a sterile environment in a laboratory. Such a process is time consuming, resulting in a delay in providing results to patients. Additionally, such tests require a patient to visit a location where a sample may be collected and transported in a sterile manner to an appropriate processing location. Travel to and from locations may risk spread of the disease being tested for and may inadvertently expose medical personnel to the disease.

In view of the above, the inventors have recognized the benefits of a detection component of a rapid diagnostic test that is usable by a user who may not be a trained medical professional. In particular, the inventors have appreciated that a user who is not a trained medical professional may not be adept at mixing a sample solution and diluent in appropriate concentrations for running on a readout element (e.g., a lateral flow assay strip). In some cases, improper mixing of diluent and sample may result in obscured or difficult to read test results, which may not be readily perceptible to an untrained user. Accordingly, the inventors have recognized the benefits of an absorbent pad containing a diluent or other fluid configured to mix with a fluid sample while maintaining sterility. For example, the inventors have recognized the benefits of an absorbent pad at least partially saturated with a diluent, which may mix with a fluid sample when the fluid sample flows toward a readout element or other fluidic component. Such an arrangement may ensure that a readout from a readout element (e.g., lateral flow assay strip) is readily apparent to a user of the absorbent pad. Accordingly, such an absorbent pad may allow users to perform tests at home and receive results in a rapid manner without necessarily requiring input from trained medical staff. Telemedicine or applications on a personal device may be employed to further enhance the usability of a rapid diagnostic test and the detection component, such that a variety of diseases such as COVID-19, influenza, (or any target nucleic acid) may be tested for in a home environment. Of course, a diagnostic test according to exemplary embodiments described herein may be administered by trained medical staff in an at-home setting or in a point-of-care setting, as the present disclosure is not so limited.

### Absorbent Pad for Diagnostic Test

According to exemplary embodiments described herein, an absorbent pad for a diagnostic test may be composed of an absorbent material configured to be disposed between two fluidic components of a diagnostic test. For example, in some exemplary embodiments described herein, an absorbent pad may be configured to be disposed between a receptacle configured to receive a sample and a readout element. The absorbent pad may be at least partially saturated with a first solution that is configured to mix with other fluids flowing between the two fluidic components of a diagnostic test. That is, the absorbent pad may be saturated below a threshold saturation such that the absorbent pad does not allow the first solution to escape the absorbent pad. However, when additional fluid is introduced to the absorbent pad, the saturation may exceed a threshold saturation such that fluid is able to flow out of the absorbent pad. The absorbent pad may ensure the first solution and the introduced fluid are appropriately mixed at desired concentrations. In some embodiments, the absorbent pad may ensure the concentration of the first solution is greater than that of the introduced fluid as a mixture flows out of the absorbent pad. Absorbent pads may be employed in any component of a diagnostic test to form a fluid coupling between two fluidic components, as the present disclosure is not so limited. It should be noted that any specific detection component or diagnostic test including an absorbent pad shown and described herein is exemplary.

In some embodiments, testing a sample on a lateral flow assay strip may generate one or more signal bands that indicate whether a target nucleic acid sequence is present in the sample. In some cases, the brightness of these signal bands may be at least partly determined based on how much a sample is diluted before being passed through the lateral flow assay strip. If a sample is not diluted prior to passing through the lateral flow assay strip, the signal bands may be dim and accordingly hard to perceive for an at-home user of a diagnostic test. Accordingly, the inventors have recognized the benefits of an absorbent pad containing a diluent that is configured to receive and dilute a sample before that sample flows to a lateral flow assay strip. In some embodiments, the absorbent pad may be at least partially saturated with diluent, where the saturation of the pad is below a saturation threshold where the lateral flow assay strip is configured to run. When a reservoir containing a sample (e.g., a vial) is fluidly connected to the absorbent pad, the absorbent pad may receive the sample and the sample will mix with the diluent. The saturation of the diluent pad once the sample is received may be greater than the threshold saturation for the lateral flow assay strip to run. Accordingly, the lateral flow assay strip may not run until the sample is received, and the sample mixes with the diluent on the absorbent pad. In other embodiments, an absorbent pad may be configured to receive a diluent from a dropper or other dispensing device prior to receiving a sample. In such an embodiment, the absorbent pad may be fully saturated by the diluent, so long as the sample is received within a threshold time period so as to be included in the lateral flow assay strip run. Other arrangements are also contemplated, as will be discussed further herein.

### Diagnostic Test

The present disclosure provides diagnostic devices, systems, and methods for rapidly (and in an at-home environment) detecting one or more target nucleic acid sequences (e.g., a nucleic acid sequence of a pathogen, such as SARS-CoV-2 or an influenza virus) that employ one or more absorbent pads containing one or more solutions. That is, various exemplary detection components and diagnostic test kits described herein may employ one or more absorbent pads containing one or more solutions that assist with sequential mixing and/or flow of fluids during a diagnostic testing process. A diagnostic system, as described herein, may be self-administrable and comprise a sample-collecting component (e.g., a swab) and a diagnostic device. The diagnostic device may comprise a cartridge, a blister pack, and/or a “chimney” detection component, according to some embodiments. In some cases, the diagnostic device comprises a detection component (e.g., a lateral flow assay strip), results of which are self-readable, or automatically read by a computer algorithm. In certain embodiments, the diagnostic device further comprises one or more reagents (e.g., lysis reagents, nucleic acid amplification reagents, CRISPR/Cas detection reagents). In certain other embodiments, the diagnostic system separately includes one or more reaction tubes comprising the one or more reagents. The diagnostic device may also comprise an integrated heater, or the diagnostic system may comprise a separate heater. The isothermal amplification technique employed yields not only fast but very accurate results. Several examples of diagnostic tests including an absorbent pad follow below.

### Diagnostic Test Detection Component Including Absorbent Pad

The inventors have also appreciated the benefits of a detection component including an absorbent pad. In some embodiments, the detection component may include a readout element (e.g., a lateral flow assay strip), an absorbent pad, and a receptacle. The absorbent pad may be disposed in a fluidic channel between the receptacle and the readout element, and may be at least partially saturated with a first solution (e.g., a diluent). The absorbent pad may have an average pore size configured to prevent liquid from flowing to the readout element until a threshold saturation is reached. The receptacle may be configured to receive a fluid sample (e.g., from a reaction tube, vial, pipette, etc.) which may flow toward the readout element via the fluidic channel. The sample may mix with the first solution on the absorbent pad. The introduction of the sample to the absorbent pad may saturate the absorbent pad above a threshold saturation, such that the mixture of sample and first solution flows to the readout element. Such an arrangement may ensure the sample is reliably mixed with the first solution.

In some cases, it may be desirable to provide a detection component separate from other portions of a diagnostic test. For example, various components to take a sample and prepare the sample for detection in the detection component may be separate from the detection component. In some embodiments, a detection component may include a lateral flow assay strip configured to receive a prepared sample to determine if one or more target nucleic acid sequences are present in the prepared sample. In some cases, as noted above, it may be desirable for a prepared sample to be mixed with a diluent prior to flowing through the lateral flow assay strip. Accordingly, in some embodiments, a detection component may include an absorbent pad containing a diluent configured to mix with a prepared sample contained in an external reservoir (e.g., a vial). The detection component may include a seal, such as a puncturable seal, that fluidly seals the absorbent pad until the sample is received by the detection component. Accordingly, the seal may prevent the diluent from evaporating or otherwise moving in the detection component, so that the detection component may be transported and/or stored with an at least partially saturated absorbent pad. In some embodiments, the act of fluidly connecting the sample reservoir to the detection component may open the seal of the absorbent pad, thereby causing the diluent and sample to mix on the absorbent pad prior to flowing to the lateral flow assay strip. Various other embodiments will be described herein which promote the mixing of a sample and diluent in a manner that is easy to operate and sterile for an at-home user of a diagnostic system. Of course, a detection component may include any suitable absorbent pad containing any desired reagent for a detection process, as the present disclosure is not so limited.

In some embodiments, a detection component of a diagnostic test includes an absorbent pad and a lateral flow assay strip. The absorbent pad may be in fluid communication with the lateral flow assay strip and may contain a diluent. The absorbent pad may be saturated by the diluent to a level below a threshold saturation where the lateral flow assay strip runs. That is, the absorbent pad may have an average pore size configured to prevent liquid from flowing to a lateral flow assay strip until the threshold saturation is reached. Accordingly, the lateral flow assay strip may not inadvertently run with just the diluent in the absorbent pad. In some embodiments, absorbent pad and lateral flow assay strip may be disposed in a housing. In such an embodiment, the detection component may include a receptacle configured to receive a first reservoir (e.g., a vial) containing a sample. The absorbent pad and receptacle may be configured so that when the first reservoir is moved into the receptacle, the sample may flow toward the absorbent pad. In this manner, the movement of the first reservoir into the receptacle may function to fluidly connect the first reservoir to the housing. Accordingly, in a single action of inserting the first reservoir into the receptacle, a user may fluidly connect the first reservoir to the absorbent pad to allow the diluent and sample to mix on the absorbent pad. In some embodiments, a seal disposed in the housing may be opened by the insertion of the first reservoir into the receptacle.

According to exemplary embodiments described herein, a seal of a detection component may be formed of a frangible material, such that the seal may be punctured or otherwise destructively broken to be opened. For example, a puncturable seal may be a breakable metal foil, a breakable film such as a plastic film or an elastomeric film that is puncturable. In some embodiments, the seal may be positioned in a fluid channel between a receptacle opening and an absorbent pad. According to one such embodiment, insertion of a first reservoir (e.g., a vial) containing a sample into a receptacle of the detection component may puncture the seal. For example, in some embodiments, the insertion of the first reservoir may crush the seal. As another example, the insertion of the first reservoir into the receptacle may pressurize a fluid channel in the detection component until a threshold pressure is reached, whereupon the seal is punctured by the pressure. In some embodiments, the first reservoir may interact with an actuator when the first reservoir is inserted into the receptacle. For example, the first reservoir may depress a lever or plunger which may in turn open the seal. Of course, any suitable arrangement for a detection component including a seal may be employed, as the present disclosure is not so limited.

According to exemplary embodiments described herein, a seal of a detection component may be configured as a valve. The valve may be switched between a closed state where an absorbent pad is sealed from the surrounding environment or adjacent reservoirs and an open state where the absorbent pad is unsealed from the surrounding environment or adjacent reservoirs. In some embodiments, movement of a first reservoir into a receptacle of the detection component may open the valve. In some embodiments, the valve may be configured as a ball valve, flutter valve, umbrella valve, pinch valve, septum valve, or any other suitable valve that may interact with a first reservoir as the first reservoir is moved into a receptacle. In some embodiments, a detection component may include an actuator coupled to the valve configured to open and/or close the valve when the first reservoir is inserted into a receptacle of the detection component. For example, the actuator may be a lever or a plunger that is moved by the first reservoir to switch the valve from a closed state to an open state. Of course, any suitable actuator may be used to open or close a valve, as the present disclosure is not so limited.

In some embodiments, a diagnostic test kit includes a first reservoir (e.g., a vial), a dropper, and a detection component. The detection component may include an absorbent pad and a lateral flow assay strip. The absorbent pad may be in fluid communication with the lateral flow assay strip and may be configured to receive a diluent. The diluent may be disposed in the dropper, which may be used to dispense the diluent into the detection component, at least partially saturating the absorbent pad. In some embodiments, the absorbent pad and lateral flow assay strip may be disposed in a housing. In such an embodiment, the detection component may include a receptacle configured to receive the first reservoir, which may contain a sample. According to this embodiment, the diluent may also be received via the receptacle. The absorbent pad and receptacle may be configured so that when the first reservoir is moved into the receptacle, the sample may flow toward the absorbent pad. In this manner, the movement of the first reservoir into the receptacle may function to fluidly connect the first reservoir to the housing. Accordingly, a user may first dispense the diluent into the detection component to saturate the absorbent pad, and subsequently a user may fluidly connect the first reservoir to the absorbent pad to allow the diluent and sample to mix on the absorbent pad.

According to exemplary embodiments described herein, an absorbent pad of a detection component may be formed of any suitable absorbent material. In some embodiments, the absorbent pad may be formed of cotton. Of course, an absorbent pad may be formed of any suitable material, including, but not limited to, cellulosic materials (e.g., nitrocellulose), glass fiber, and polyester. An absorbent pad may have any suitable dimensions (e.g., thickness, width, height, etc.) for absorbing a fluid and transferring that solution to a lateral flow assay strip. In some embodiments, an absorbent pad may have an average pore size configured to prevent liquid from flowing to a lateral flow assay strip until a threshold fill level (e.g., saturation) is reached. That is, the average pore size may be sufficiently small so as to prevent liquid from flowing until a sample is introduced to the absorbent pad. Once the absorbent pad reaches a threshold fill level, the liquid in the absorbent pad wets out a glass fiber matrix of the absorbent pad and eventually make fluidic connected with a lateral flow assay strip.

While exemplary embodiments described herein relate to saturation of an absorbent pad containing a diluent solution, it should be understood that the techniques described herein may be applied to any suitable solution. An absorbent pad may contain any desired solution including any number of reagents for a diagnostic test, as the present disclosure is not so limited.

Additionally, while exemplary embodiments described herein relate to a detection component including an absorbent pad, it should be understood that absorbent pads according to exemplary embodiments described herein may be included in any portion of a diagnostic testing system. One or more absorbent pads may be employed in a detection component (e.g., cartridge, “chimney”, blister pack) or any other suitable component of a diagnostic testing system, as the present disclosure is not so limited.

### Diagnostic Test Detection Component Including Receptacle and Absorbent Pad

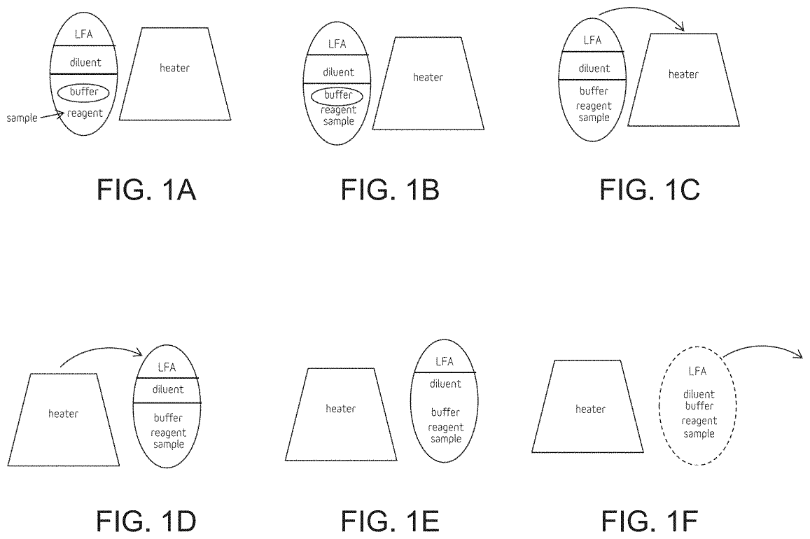

FIGS. 1A-1F show, according to some embodiments, a general process of performing a diagnostic test for the presence of one or more nucleic acid sequences. As shown in FIG. 1A, a sample is added to a first reservoir containing one or more reagents (e.g., lysis reagents, nucleic acid amplification reagents, CRISPR/Cas detection reagents). The one or reagents may react with the sample to begin a diagnostic testing process. As shown in FIG. 1A, a second reservoir containing a diluent may be kept separately from the first reservoir containing the reagents. As shown in FIG. 1B, the sample and reagent may mix for a predetermined time period. A buffer may also be added into the first reservoir, such that a mixture of the sample, one or more reagents, and buffer are contained in the first reservoir. As shown in FIG. 1C, the mixture in the first reservoir may be heated in a heater or by another appropriate method such as an exothermic chemical reaction. Once heated as shown in FIGS. 1D-1E, the heated mixture contained in the first reservoir may be subsequently mixed with the diluent solution contained in the second reservoir. Once the diluent and sample mixture have mixed, the combined diluent and sample may be exposed to a lateral flow assay (LFA) strip, which may indicate the results of the diagnostic test. Following the reading of the LFA strip, one or more disposable components may be disposed of, as shown in FIG. 1F.

As shown in FIGS. 1A-1F, the process of performing a diagnostic test includes multiple steps of fluid combination at different times. Furthermore, additional steps such as heating are also performed through the testing process. According to exemplary embodiments described herein, the inventors have appreciated at least partially separating a detection component from other steps in the diagnostic testing process. That is, the steps shown in FIGS. 1D-1F may be accomplished using a detection component that simplifies the combination of a diluent and a sample mixture prior to being exposed to a lateral flow assay strip. Such arrangements may ensure that readings on a lateral flow assay strip are clear and well-defined, so that they may be more easily perceived by an at-home user of a diagnostic testing system.

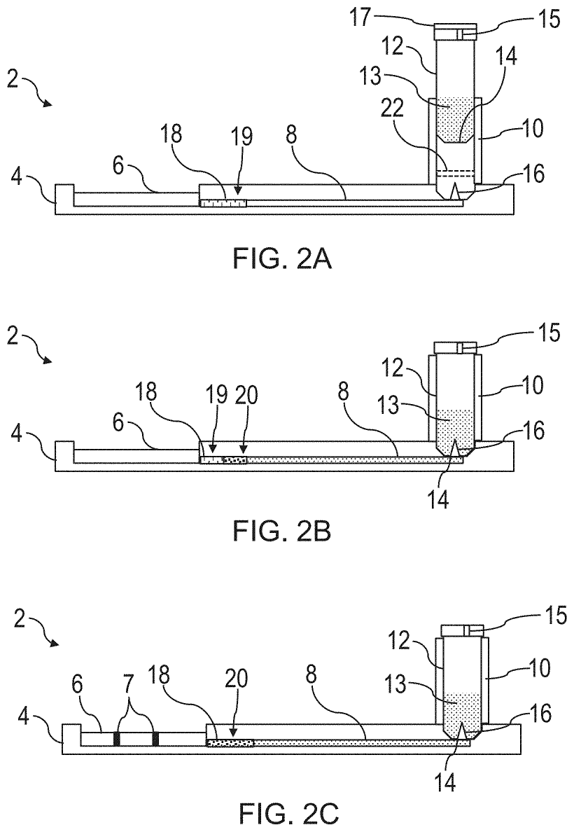

FIGS. 2A-2C show, according to some embodiments, a detection component 2 of a diagnostic test configured to ensure appropriate mixing of a sample mixture 13 and a diluent 19 prior to exposure to a lateral flow assay strip 6. As shown in FIG. 2A, the detection component includes a housing 4 containing the lateral flow assay strip 6, an absorbent pad 18, and a fluidic channel 8. The absorbent pad is in fluid communication with the lateral flow assay strip. The fluidic channel 8 fluidly connects the absorbent pad 18 to a receptacle 10. According to the embodiment of FIGS. 2A-2C, the receptacle is configured to receive a first reservoir 12 (e.g., a vial) so that the first reservoir may be fluidly connected to the fluidic channel 8. The first reservoir 12 may be slidably disposed in the receptacle 10, such that the first reservoir may be moved into the receptacle to be punctured by a blade 16 so that the first reservoir may be brought into fluid communication with the fluidic channel 8. In particular, the blade 16 is configured to puncture a bottom portion 14 of the first reservoir to fluidly connect the first reservoir to the fluidic channel 8 and correspondingly allow the sample mixture 13 to flow into the fluidic channel toward the lateral flow assay strip. Of course, in other embodiments, a needle or another suitable puncturing component or other fluidic connection may be employed, as the present disclosure is not so limited.

In some embodiments as shown in FIGS. 2A-2C, the first reservoir 12 includes a semi-permeable vent 15. The semi-permeable vent may allow the sample mixture 13 to exit the first reservoir more easily into the fluidic channel 8 compared to a reservoir without a semi-permeable vent. That is, the semi-permeable vent may allow air to enter the first reservoir as the sample mixture flows out of the first reservoir to replace any space vacated by the sample mixture. Accordingly, the semi-permeable vent may mitigate the effects of any vacuum formed in a headspace inside of the first reservoir above the sample mixture 13. The semi-permeable vent may be air-permeable but not liquid permeable, such that the sample mixture 13 is not able to flow out of the semi-permeable vent and no liquids or other contaminants are able to flow into the first reservoir. Of course, in some embodiments no additional vent may be provided into the first reservoir, as the present disclosure is not so limited. In some embodiments as shown in FIGS. 2A-2C, the first reservoir includes a removable cover 17 configured to seal the semi-permeable vent. The removable cover may prevent any air from entering the first reservoir or escaping from the first reservoir. Such an arrangement may be beneficial during a heating process of the first reservoir. In some embodiments, the removable cover may be removed from the first reservoir by peeling the removable cover off. Of course, any suitable cover may be employed and removed in any suitable manner, as the present disclosure is not so limited.

According to the embodiment of FIGS. 2A-2C, the detection component 2 includes an absorbent pad 18 containing a diluent 19. That is, the absorbent pad may be at least partially saturated with the diluent. A saturation of the absorbent pad in the initial state as shown in FIG. 2C may be less than a threshold saturation at which the lateral flow assay strip 6 runs. As noted previously, before the sample mixture 13 is bought into fluid communication with the lateral flow assay strip, it is desirable to sufficiently dilute the sample mixture to ensure the readout from the lateral flow assay strip is clear. Accordingly, the detection component 2 of FIGS. 2A-2C is configured to allow the sample and diluent to mix on the absorbent pad 18. In the arrangement of FIGS. 2A-2C, the concentration of the diluent may be greatest during an initial running of the lateral flow assay strip 6. This is a result of the absorbent pad being pre-saturated with the diluent, prior to receiving the sample mixture 13 from the first reservoir. As will be discussed further below, when the absorbent pad 18 is at least partially saturated with the sample mixture 13, the lateral flow assay strip 6 may run. However, the relative concentration of the diluent may be greater than that of the sample mixture. Such an arrangement ensures that the sample mixture is properly diluted, and may result in clearer, easier to interpret test results displayed on the lateral flow assay strip.

As shown in FIG. 2A, the detection component may include an optional seal 22 positioned between the receptacle 10 and the absorbent pad 18. According to the embodiment of FIG. 2A, the seal 22 is a frangible seal (e.g., a metal foil, frangible elastomer, etc.) that is configured to open once the first reservoir is fluidly connected to the detection component. The insertion of the first reservoir 12 into the receptacle 10 applies force to the seal 22, thereby breaking the seal and unsealing the absorbent pad 18. Such an arrangement may ensure that the diluent on the absorbent pad 18 does not evaporate or otherwise flow out of the detection component 2 prior to a detection process.

The process of using the detection component 2 to perform a diagnostic test is shown through the states of FIGS. 2A-2C. FIG. 2A is a starting state where the first reservoir 12 is not fluidly connected to the absorbent pad 18. Correspondingly, the optional seal 22 is closed and the diluent 19 is contained within the absorbent pad 18. The fluidic channel 8 is empty in the state of FIG. 2A. In the state of FIG. 2B, the first reservoir 12 has been pushed further into the receptacle 10 toward the blade 16. Accordingly, the bottom portion 14 of the first reservoir has been pierced by the blade 16, which has fluidly connected the first reservoir to the fluid channel 8. Additionally, the force applied to the first reservoir has opened the seal 22. When the first reservoir is fluidly connected to the receptacle, the sample mixture may flow automatically (e.g., under the effect of gravity, by capillary action, etc.) toward the absorbent pad. As shown in FIG. 2B, the removable cover has been removed from first reservoir to allow the semi-permeable vent 15 to vent the headspace in the first reservoir. As shown in FIG. 2B, the sample mixture 13 and diluent 19 begin to mix on the absorbent pad 18 to form a mixture 20. Once a threshold saturation of the absorbent pad is reached, the lateral flow assay strip 6 may begin to run. The absorbent pad may be formed of a material having an average pore size configured to prevent liquid from flowing to the lateral flow assay strip 6 until the threshold saturation is reached. As the lateral flow assay strip runs, the mixture 20 may have a concentration of diluent greater than a concentration of sample mixture 13, due to the physical position of the absorbent pad in a fluid path between the fluidic channel 8 and the lateral flow assay strip 6.

As shown in FIG. 2C, the sample mixture 13 continues to mix with the diluent on the absorbent pad 18, and the mixture 20 is run through the lateral flow assay strip 6. As noted previously, diluent 19 is present in the absorbent pad 18 in fluid communication with the lateral flow assay strip 6 prior to the sample mixture 13. Such an arrangement ensures that a concentration of the diluent relative to the sample is higher when the sample first encounters the lateral flow assay strip 6. The higher concentration of diluent initially may assist the lateral flow assay strip is generating definitive signal lines 7 that may be easily perceived by a user.

While in the embodiment of FIGS. 2A-2C the absorbent pad 18 is a sample pad in direct fluid communication with the lateral flow assay strip 6, in other embodiments the detection component may include a sample pad positioned elsewhere in the fluid channel 8. Of course, any suitable arrangement for a lateral flow assay strip and absorbent pad may be employed, as the present disclosure is not so limited.

According to the embodiment of FIGS. 2A-2C, the first reservoir 12 is configured to contain a sample. In some embodiments, the first reservoir may be configured to receive a sample swab (not shown), where the swab may be configured to collect a sample from a subject. The first reservoir 12 may contain a buffer solution and/or a lysis solution configured to react with the sample so that one or more target nucleic acid sequences may be detected by the lateral flow assay strip. The first reservoir 12 may be sized and shaped to fully receive the swab. Accordingly, a sample swab may be easily deposited in the first reservoir 12, and once the swab is deposited in the first reservoir 12, the first reservoir may be sealed with a cap and allowed to incubate before the bottom portion 14 of the first reservoir 12 is punctured. In some embodiments, the first reservoir may be formed as a plastic vial. Of course, the first reservoir may have any suitable construction, as the present disclosure is not so limited.

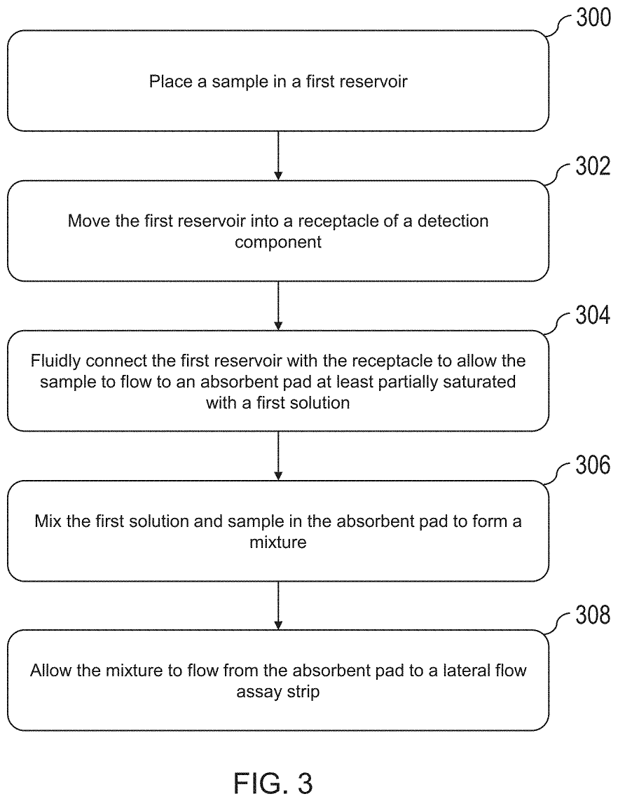

FIG. 3 shows, according to some embodiments, a flow chart for a method of performing a diagnostic test. In block 300, a sample is placed in a first reservoir, such as a vial. Placing the sample in the first reservoir may include placing a swab in the reservoir, in addition to one or more reagents. In other embodiments, a completed sample mixture may be placed in the first reservoir. In block 302, the first reservoir is moved into a receptacle of a detection component. For example, the first reservoir may be slid into a receptacle of the detection component such that the receptacle receives the first reservoir. In block 304, the first reservoir is fluidly connected with the receptacle to allow the sample contained inside of the first reservoir to flow to an absorbent pad that is at least partially saturated with a first solution. The first solution may be a diluent. Fluidly connecting the first reservoir to the receptacle may include puncturing the first reservoir with a puncturing tool, such as a blade or a needle. In other embodiments, the first reservoir may be fluidly connected to the receptacle using a fluid connector (e.g., a quick connect fluid connector). In block 306, the first solution from the first reservoir and the sample are mixed in the absorbent pad, where the absorbent pad is positioned between a lateral flow assay strip and the first reservoir. In block 308, the mixture of sample and the first solution are allowed to flow from the absorbent pad to the lateral flow assay strip. In some embodiments, the addition of the sample mixture to the sample pad may increase the saturation of the absorbent pad above a threshold saturation to run the lateral flow assay strip.

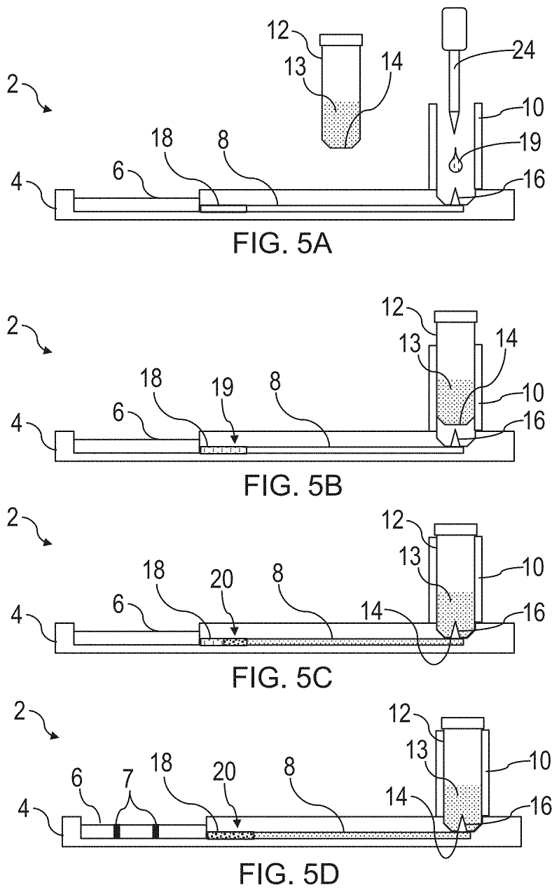

FIGS. 5A-5D depict, according to some embodiments, a detection component 2 of a diagnostic test configured to ensure appropriate mixing of a sample mixture 13 and a diluent 19 prior to exposure to a lateral flow assay strip 6. As shown in FIG. 2A, the detection component includes a housing 4 containing the lateral flow assay strip 6, an absorbent pad 18, and a fluidic channel 8. The absorbent pad is in fluid communication with the lateral flow assay strip. The fluidic channel 8 fluidly connects the absorbent pad 18 to a receptacle 10. According to the embodiment of FIGS. 5A-5D, the receptacle is configured to receive a first reservoir 12 (e.g., a vial) so that the first reservoir may be fluidly connected to the fluidic channel 8. In particular, the blade 16 is configured to puncture a bottom portion 14 of the first reservoir to fluidly connect the first reservoir to the fluidic channel 8 and correspondingly allow the sample mixture 13 to flow into the fluidic channel toward the lateral flow assay strip. Of course, in other embodiments, a needle or another suitable puncturing component or other fluidic connection may be employed, as the present disclosure is not so limited. As shown in FIG. 5A, a diagnostic test may also include a dropper 24 or another suitable dispensing device. The detection component 2 may be configured to receive a diluent 19 from the dropper 24 via the receptacle 10. Accordingly, in the embodiment of FIGS. 5A-5D, the absorbent pad 18 may not be pre-saturated with a diluent. Instead, a user may dispense the diluent 19 and saturate the absorbent pad 18 prior to fluidly connecting the first reservoir 12.

The process of using the detection component 2 to perform a diagnostic test is shown through the states of FIGS. 5A-5D. FIG. 5A is a starting state where the first reservoir 12 is not fluidly connected to the absorbent pad 18. In the state of FIG. 5A, the absorbent pad does not include any fluid. Likewise, the fluidic channel 8 is empty in the state of FIG. 5A. As shown in FIG. 5A, the dropper 24 may be used by a user to dispense a diluent into the receptacle 10 to initiate a detection process. In the state of FIG. 5B, the diluent 19 dispensed from the dropper 24 has been absorbed by the absorbent pad 18. In some embodiments, the absorbent pad 18 may have a saturation lower than a threshold saturation to initiate running the lateral flow assay strip. According to one such embodiment, the lateral flow assay strip 6 may not run until the absorbent pad 18 also receives the sample mixture 13. In other embodiments, the absorbent pad may have a saturation above a threshold saturation to initiate running the lateral flow assay strip 6. In some such embodiments, a user may fluidly connect the first reservoir 12 within a threshold time period to ensure the sample mixture 13 reaches the lateral flow assay strip while the strip is running.

In the state of FIG. 5C, the first reservoir 12 has been pushed into the receptacle 10 toward the blade 16. Accordingly, the bottom portion 14 of the first reservoir has been pierced by the blade 16, which has fluidly connected the first reservoir to the fluid channel 8. As shown in FIG. 5C, the sample mixture 13 and diluent 19 begin to mix on the absorbent pad 18 to form a mixture 20. The lateral flow assay strip 6 may begin to run or may continue to run with the mixture 20 to determine is a target nucleic acid sequence is present. As the lateral flow assay strip runs, the mixture 20 may have a concentration of diluent greater than a concentration of sample mixture 13, due to the physical position of the absorbent pad between the fluidic channel 8 and the lateral flow assay strip 6.

As shown in FIG. 5D, the sample mixture 13 continues to mix with the diluent on the absorbent pad 18, and the mixture 20 is run through the lateral flow assay strip 6. As noted previously, diluent 19 is present in the absorbent pad 18 in fluid communication with the lateral flow assay strip 6 prior to the sample mixture 13. Such an arrangement ensures that a concentration of the diluent relative to the sample is higher when the sample first encounters the lateral flow assay strip 6. The higher concentration of diluent initially may assist the lateral flow assay strip is generating definitive signal lines 7 that may be easily perceived by a user.

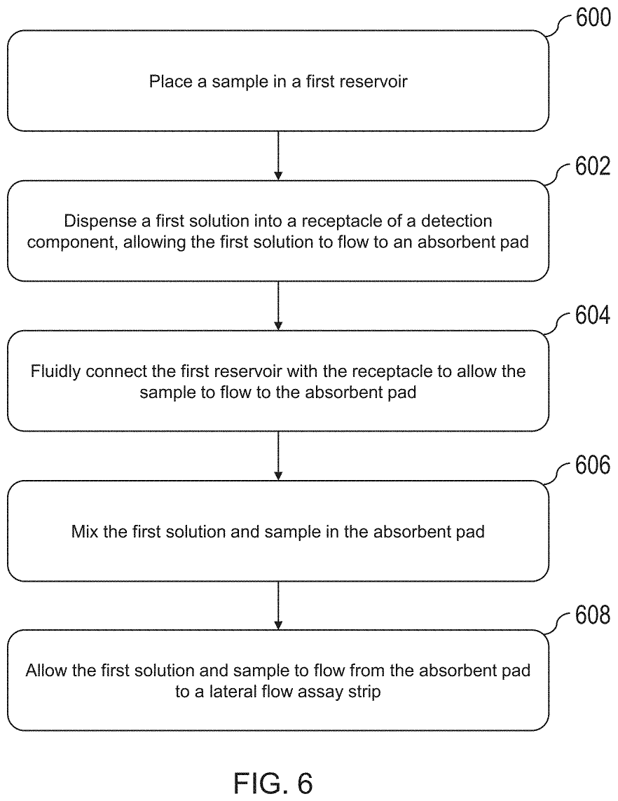

FIG. 6 shows, according to some embodiments, a flow chart for a method of performing a diagnostic test. In block 600, a sample is placed in a first reservoir, such as a vial. Placing the sample in the first reservoir may include placing a swab in the reservoir, in addition to one or more reagents. In other embodiments, a completed sample mixture may be placed in the first reservoir. In block 602, a first solution is dispensed into a receptacle of a detection component, allowing the first solution to flow to an absorbent pad. In some embodiments, the first solution may be a diluent. In some embodiments, the first solution may be dispensed with a dropper or a pipette. In block 604, the first reservoir is moved into a receptacle of a detection component and fluidly connected with the receptacle. For example, the first reservoir may be slid into a receptacle of the detection component. When the first reservoir is fluidly connected with the receptacle, the sample contained inside of the first reservoir may flow to an absorbent pad that is at least partially saturated with the first solution. Fluidly connecting the first reservoir to the receptacle may include puncturing the first reservoir with a puncturing tool, such as a blade or a needle. In other embodiments, the first reservoir may be fluidly connected to the receptacle using a fluid connector (e.g., a quick connect fluid connector). In block 606, the first solution from the first reservoir and the sample are mixed in the absorbent pad, where the absorbent pad is positioned between a lateral flow assay strip and the first reservoir. In block 608, the mixture of sample and the first solution are allowed to flow from the absorbent pad to the lateral flow assay strip. The mixture of the first solution and sample may ensure that signal lines on the lateral flow assay strip are visible and well-defined.

### Method of Manufacturing a Diagnostic Test Including an Absorbent Pad

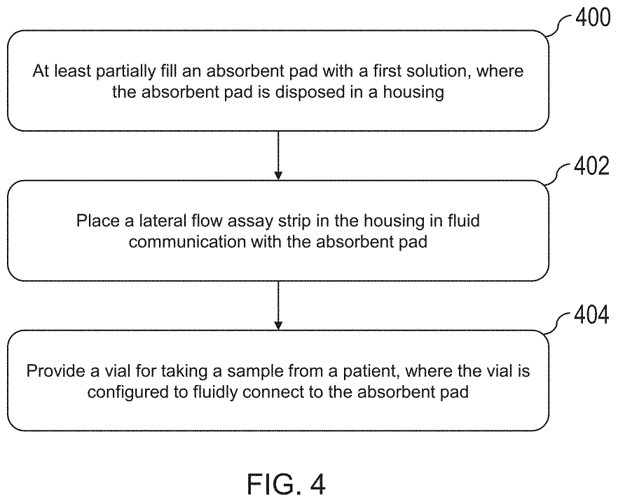

FIG. 4 shows, according to some embodiments, a flow chart for a method of manufacturing a diagnostic test including an absorbent pad. In block 400, an absorbent pad is at least partially filled with a first solution, where the first reservoir is disposed in a housing. In some embodiments, the absorbent pad may be saturated to a level below a threshold saturation for running a lateral flow assay strip. In block 402, a lateral flow assay strip is placed in the housing in fluid communication with the absorbent pad. In block 404, a vial is provided for taking a sample from a patient. The vial may be configured to fluidly connect to the housing (e.g., via a receptacle). The action of fluidly connecting the vial to the housing may allow fluid to flow from the vial to the absorbent pad. The process shown in FIG. 4 may be employed to make a diagnostic test similar to that shown and described with reference to FIGS. 2A-2C.

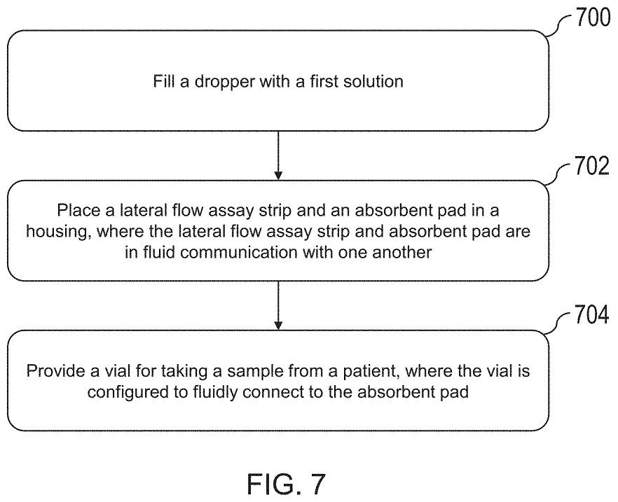

FIG. 7 shows, according to some embodiments, a flow chart for a method of manufacturing a diagnostic test including an absorbent pad. In block 700, a dropper is filled with a first solution. The first solution may be a diluent. In block 702, a lateral flow assay strip and absorbent pad are placed in the housing in fluid communication with one another. In block 704, a vial is provided for taking a sample from a patient. The vial may be configured to fluidly connect to the housing (e.g., via a receptacle). The action of fluidly connecting the vial to the housing may allow fluid to flow from the vial to the absorbent pad. The process shown in FIG. 7 may be employed to make a diagnostic test similar to that shown and described with reference to FIGS. 5A-5D.

### “Chimney” Detection Component Including an Absorbent Pad

In some embodiments, a diagnostic device comprises a detection component comprising a “chimney.” In certain embodiments, the “chimney” detection component comprises a chimney configured to receive a reaction tube. In certain embodiments, the “chimney” detection component comprises a puncturing component configured to puncture the reaction tube. The puncturing component may comprise one or more blades, needles, or other elements capable of puncturing a reaction tube. In certain embodiments, the “chimney” detection component comprises a lateral flow assay strip. As described herein, the lateral flow assay strip may comprise one or more test lines configured to detect one or more target nucleic acid sequences. In some embodiments, the lateral flow assay strip further comprises one or more control lines.

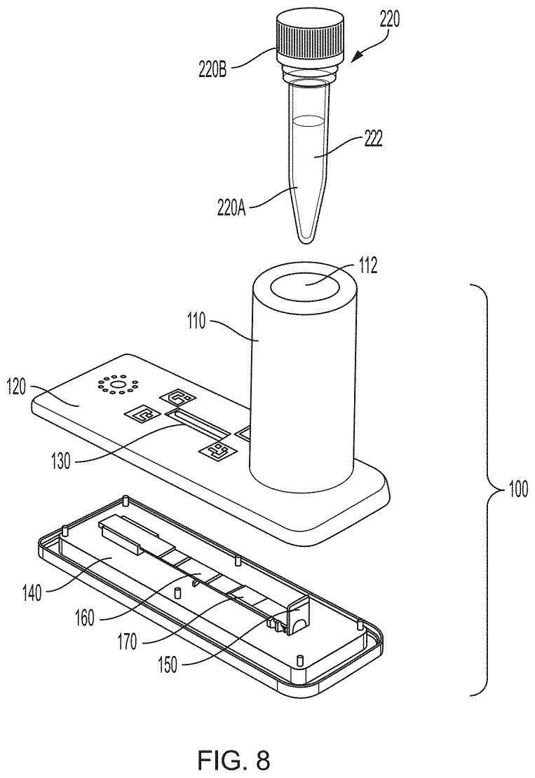

One embodiment of a “chimney” detection component is shown in FIG. 8. In FIG. 8, detection component 100 comprises chimney 110, front panel 120 comprising opening 130, and back panel 140 comprising puncturing component 150 and lateral flow assay strip 160. In some embodiments, chimney 110 and front panel 120 are integrally formed. In some embodiments, chimney 110 and front panel 120 are separately formed components that are attached to each other (e.g., via one or more screws or other fasteners, one or more adhesives, and/or one or more interlocking components). In some embodiments, front panel 120 and back panel 140 are attached to each other (e.g., via one or more screws or other fasteners, one or more adhesives, and/or one or more interlocking components). In some embodiments, front panel 120 comprises one or more markings (e.g., ArUco markers) to facilitate alignment of an electronic device (e.g., a smartphone, a tablet) with opening 130.

In operation, a reaction tube comprising fluidic contents may be inserted into an opening 112 of the chimney 110. In some embodiments, the reaction tube comprises a cap (e.g., a screw-top cap, a hinged cap) and a bottom end (e.g., a tapered or rounded bottom end). In certain cases, as shown in FIG. 8, the bottom end of the reaction tube is inserted into chimney 110 prior to the cap of the reaction tube. In certain cases, the reaction tube is inverted, and the cap of the reaction tube is inserted into chimney 110 prior to the bottom end of the reaction tube. In some embodiments, upon insertion into chimney 110, the reaction tube may lock or snap into place (or may otherwise have a secure fit) such that the reaction tube may not be easily removed from chimney 110 by the user. In certain cases, locking or snapping the reaction tube into place (or otherwise preventing easy removal of the reaction tube from chimney 110) may reduce or prevent contamination.

In some embodiments, the reaction tube may be punctured by puncturing component 150. As a result, at least a portion of the fluidic contents of the reaction tube may be deposited on a first sub-region (e.g., an absorbent pad 170) of lateral flow assay strip 160. In some cases, at least a portion of the fluidic contents of the reaction tube may be transported through lateral flow assay strip 160 (e.g., via capillary action). In some cases, for example, at least a portion of the fluidic contents of the reaction tube may flow through a second sub-region (e.g., a particle conjugate pad) of lateral flow assay strip 160 comprising a plurality of labeled particles. In some instances, the fluidic contents of the reaction tube may comprise one or more amplified nucleic acids (e.g., amplicons), and flow of at least a portion of the fluidic contents through the second sub-region (e.g., particle conjugate pad) of lateral flow assay strip 160 may result in one or more labeled amplicons. In some cases, at least a portion of the fluidic contents of the reaction tube (which may, in some instances, comprise one or more labeled amplicons) may flow through a third sub-region (e.g., a test pad) comprising one or more test lines comprising one or more capture reagents (e.g., immobilized antibodies) configured to detect one or more target nucleic acid sequences. In some instances, the formation (or lack of formation) of one or more opaque lines may indicate the presence or absence of one or more target nucleic acid sequences. In certain cases, the one or more opaque lines (if present) may be visible through opening 130 of front panel 120.

As shown in FIG. 8, the detection component 100 also includes an absorbent pad 170 configured to contain a diluent solution. According to the embodiment of FIG. 8, the absorbent pad may be at least partially saturated with a diluent prior to the connection of the tube 220A to the detection component. In some embodiments, the absorbent pad 170 may be at least partially saturated with a diluent during a manufacturing process. In other embodiments, a dropper or other dispenser may be employed to dispense diluent onto the absorbent pad (e.g., via the chimney 110). The absorbent pad may be saturated to a level less than a threshold saturation to run the lateral flow assay strip 160. When tube 220A is punctured by the puncturing component 150, a sample 222 contained in the tube 220A may flow automatically (e.g., under the effect of gravity, by capillary action, etc.) to the absorbent pad and mix with the diluent. The diluent solution and sample 222 are allowed to mix prior to flowing to the lateral flow assay strip (e.g., by capillary action), thereby improving the clarity of one or more test lines on the lateral flow assay strip.

In some embodiments, a diagnostic system comprises a sample-collecting component (e.g., a swab), a reaction tube comprising one or more reagents, and a “chimney” detection component. In some embodiments, the diagnostic system further comprises a heater, as described herein.

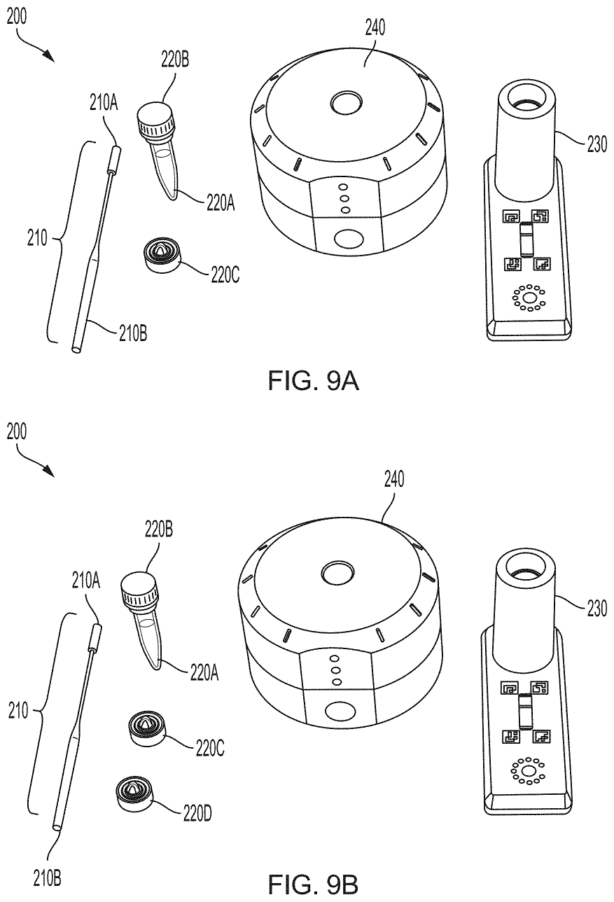

One embodiment of a diagnostic system comprising a “chimney” detection component is shown in FIG. 9A. In FIG. 9A, diagnostic system 200 comprises sample-collecting component 210, reaction tube 220, “chimney” detection component 230, and heater 240. As shown in FIG. 9A, sample-collecting component 210 may be a swab comprising swab element 210A and stem element 210B. In certain embodiment, reaction tube 220 comprises tube 220A, first cap 220B, and second cap 220C. As shown in FIG. 9A, first cap 220B and/or second cap 220C may be screw-top caps or any other types of removable caps. In certain embodiments, first cap 220B and/or second cap 220C may be airtight caps (e.g., they may fit on reaction tube 220A without any gaps and seal reaction tube 220A). In certain embodiments, second cap 220C may comprise one or more reagents (e.g., lysis reagents, nucleic acid amplification reagents, CRISPR/Cas detection reagents). In some instances, for example, second cap 220C comprises one or more blister packs comprising one or more reagents. In some embodiments, reaction tube 220 comprises fluidic contents. In certain cases, the fluidic contents of reaction tube 220 comprise a reaction buffer. In certain embodiments, the reaction buffer comprises one or more buffers (e.g., phosphate-buffered saline (PBS), Tris). In certain embodiments, the reaction buffer comprises one or more salts. Reaction tube 220 may contain any suitable volume of the reaction buffer.

In operation, a user may collect a sample using sample-collecting component 210. In some instances, for example, the user may insert swab element 210A into a nasal or oral cavity of a subject (e.g., the user, a friend or family member of the user, or any other human or animal subject). Cap 220B may be removed from tube 220A (e.g., either before or after collection of the sample), thereby exposing the fluidic contents of tube 220A, and, after collecting the sample, swab element 210A may be inserted into the fluidic contents of tube 220A. In some cases, the user may stir swab element 210A in the fluidic contents of tube 220A for a period of time (e.g., at least 10 seconds, at least 20 seconds, at least 30 seconds). In certain instances, swab element 210A is removed from tube 220A. In certain other instances, stem element 210B is broken and removed such that swab element 210A remains in tube 220A.

After swab element 210A and/or stem element 210B is removed from tube 220A, a cap may be placed on tube 220A. In some instances, for example, second cap 220C may be placed on tube 220A. In some cases, tube 220A and/or second cap 220C comprise one or more reagents (e.g., lysis reagents, nucleic acid amplification reagents, CRISPR/Cas detection reagents). In certain embodiments, second cap 220C comprises one or more reagents. In some instances, the one or more reagents are in solid form (e.g., lyophilized, dried, crystallized, air jetted). In some cases, for example, the one or more reagents are in the form of one or more tablets and/or pellets. In certain instances, the one or more tablets and/or pellets comprise one or more coatings (e.g., a coating of a time release material). In some instances, the one or more reagents are in liquid form.

The one or more reagents may be released into reaction tube 220A by any suitable mechanism. In some cases, the one or more reagents may be released into tube 220A by inverting (and, in some cases, repeatedly inverting) reaction tube 220. In some cases, second cap 220C comprises a seal (e.g., a foil seal) separating the one or more reagents from the contents of tube 220A, and the seal may be punctured by screwing second cap 220C onto tube 220A, by puncturing the seal with a puncturing tool, or otherwise puncturing the seal. In some cases, the user presses on a button or other portion of second cap 220C and/or twists at least a portion of second cap 220C to release the one or more reagents into tube 220A.

In some embodiments, reaction tube 220 may be inserted into heater 240. Reaction tube 220 may be heated at one or more temperatures (e.g., at least 37° C., at least 65° C.) for one or more periods of time. In some cases, heating reaction tube 220 according to a first heating protocol (e.g., a first set of temperature(s) and time period(s)) may facilitate lysis of cells within the collected sample. In a particular, non-limiting embodiment, a first heating protocol comprises heating reaction tube 220 at 37° C. for 5-10 minutes (e.g., about 3 minutes) and at 65° C. for 5-10 minutes (e.g., about 10 minutes). In some cases, heating reaction tube 220 according to a second heating protocol (e.g., a second set of temperature(s) and time period(s)) may facilitate amplification of one or more target nucleic acids (if present within the sample). In a particular, non-limiting embodiment, a second heating protocol comprises heating reaction tube 220 at 37° C. for 10-15 minutes. In some cases, the heater may comprise an indicator (e.g., a visual indicator) that a heating protocol is occurring. The indicator may indicate to a user when the reaction tube should be removed from the device.

Following heating, reaction tube 220 may be inserted into “chimney” detection component 230. Upon insertion, reaction tube 220 may be punctured by a puncturing component (e.g., a blade, a needle) of “chimney” detection component 230. In some cases, at least a portion of the fluidic contents of reaction tube 220 are deposited onto a portion of a lateral flow assay strip of “chimney” detection component 230. The fluidic contents of reaction tube 220 may flow through the lateral flow assay strip (e.g., via capillary action), and the presence or absence of one or more target nucleic acid sequences may be indicated on a portion of the lateral flow assay strip (e.g., by the formation of one or more lines on the lateral flow assay strip). In some instances, for example, the portion of the lateral flow assay strip may be visible to a user (e.g., through an opening, a clear window, etc.). In some cases, software (e.g., a mobile application) may be used to read, analyze, and/or report the results (e.g., the one or more lines of the lateral flow assay strip). In some embodiments, “chimney” detection component 230 comprises one or more markings (e.g., ArUco markers) to facilitate to facilitate alignment of an electronic device (e.g., a smartphone, a tablet) with “chimney” detection component 230.

FIG. 9B shows an embodiment of diagnostic system 200 comprising reaction tube 220 comprising tube 220A, first cap 220B, second cap 220C, and third cap 220D. In certain cases, second cap 220C and third cap 220D each comprise one or more reagents. In some cases, second cap 220C may contain a first set of reagents (e.g., lysis reagents), and third cap 220D may comprise a second set of reagents (e.g., nucleic acid amplification reagents). In some cases, caps may have different colors to indicate that they contain different reagents. For example, in FIG. 9B, second cap 220C is red, while third cap 220D is blue. In some cases, the first set of reagents and/or the second set of reagents are in solid form (e.g., lyophilized, dried, crystallized, air jetted). In certain cases, for example, the one or more reagents are in the form of one or more tablets and/or pellets. In certain instances, the one or more tablets and/or pellets comprise one or more coatings (e.g., a coating of a time release material). In some cases, coatings of different materials and/or thicknesses may delay release of one or more reagents to an appropriate time in the reaction and may facilitate the sequential adding of different reagents.

### Diagnostic Test Detection Component Including Blisters and Absorbent Pad

In some cases, a detection component may include one or more reservoirs configured to contain one or more reagents for a detection process. The one or more reservoirs may be configured as blisters, which may be broken or otherwise activated to transfer the reagents or other fluids throughout the detection component. Accordingly, a detection component may be configured as a blister pack. In some embodiments, such a detection component may also include an absorbent pad in fluid communication with a lateral flow assay strip. The absorbent pad may be configured to contain or otherwise receive a diluent, such that a sample to be tested on the lateral flow assay strip is sufficiently diluted. The dilution provided by the absorbent pad may ensure one or more signal lines on the lateral flow assay strip and clear and well defined, so it may be determined if a target nucleic acid is present in the sample.

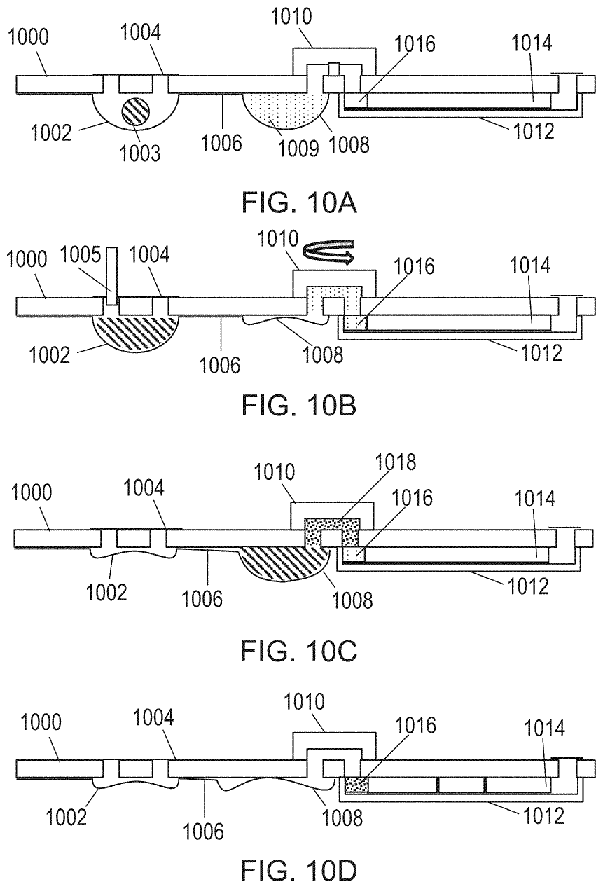

FIGS. 10A-10D depict a process of completing a diagnostic testing process using one embodiment of a diagnostic test detection component configured as a blister pack 1000. As shown in FIG. 10A, the blister pack 1000 comprises first chamber 1002, sample port 1004, seal 1006, second chamber 1008, valve 1010, third chamber 1012, lateral flow assay strip 1014, and absorbent pad 1016. According to the embodiment of FIGS. 10A-10D, the first chamber 1002 may comprise one or more amplification reagents 1003 (e.g., LAMP, RPA, NEAR reagents) in solid form (e.g., lyophilized) The second chamber 1008 comprises a diluent 1009 which is a liquid solution. The third chamber 1012 houses the lateral flow assay strip 1014. As shown in FIG. 10A, the first chamber 1002 and second chamber 1008 may be separated by a breakable seal 1006 (e.g., a frangible seal). When a threshold force is applied to the first blister chamber 1002 and/or the second blister chamber 1008, the breakable seal may be configured to open (i.e., burst). As shown in FIG. 10A, the second chamber 1008 and third chamber 1012 are separated by a rotary valve, where the valve may be rotated to open or close a fluidic channel between the second blister chamber 1008 and the third chamber 1012. That is, rotating the rotary valve may switch the valve between an open state and a closed state.

FIG. 10A may represent a state in which the diagnostic test is delivered to an end user before the diagnostic testing process begins. As shown in FIG. 10B, the first step of performing a diagnostic test may include taking a sample, and then placing that sample in the first blister chamber 1002. In particular, as shown in FIG. 10B, placing the sample in the first blister chamber includes moving a pipette 1005 through the sample port 1004. According to the embodiment of FIGS. 10A-10D, the sample port may be a septum that is non-destructively opened by the pipette 1005. As shown in FIG. 10B, the pipette 1005 may be used to deposit a liquid sample into the first blister chamber 1002. The liquid sample may react with the solid amplificant reagents 1003 shown in FIG. 10A. Of course, while a liquid sample is shown being deposited in FIG. 10B, in other embodiments a solid sample may be deposited in a blister chamber via a sample port, as the present disclosure is not so limited.

Once the sample is deposited in the first chamber 1002, the sample may be allowed to react with the amplification reagents for a predetermined amount of time. In some embodiments, the first blister chamber 1002 may be heated for a predetermined period of time (e.g., with an external heater). Once the solution inside of the first blister chamber 1002 has had a predetermined time to react, the valve 1010 may be moved to open a fluidic channel between the second blister chamber 1008 and the third chamber 1012. Force may be applied to the second blister chamber 1008 to force the diluent 1009 through the fluidic channel and into the absorbent pad 1016. The absorbent pad 1016 may absorb the buffer. In some embodiments, the saturation of the absorbent pad provided by the diluent may be less than a saturation to trigger the running of the lateral flow assay strip 1014.

Once the diluent 1009 has been transferred to the absorbent pad 1016, an external force may be applied to the first blister chamber 1002. As shown in FIG. 10C, when a threshold force is applied to the first blister chamber 1002, the breakable seal 1006 may be broken and the solution inside of the first blister chamber may be forced into the second blister chamber 1008. That is, the first blister chamber 1002 may collapse under the application of the threshold force, thereby forcing the fluid from the first blister chamber into the second blister chamber 1008. Accordingly, the seal 1006 of the embodiment of FIGS. 10A-10D is a burstable type seal, where fluid from the first blister chamber 1002 is uncontrollably released into the second blister chamber 1008. Once the combined solution of the first blister chamber is moved to the second blister chamber, the combined sample solution may mix with the diluent to form a mixture 1018. As shown in FIG. 10C, once the combined solution in the second blister chamber 1008 is ready to fully move to the lateral flow assay strip 1014, the second blister chamber 1008 may be depressed to move the solution contained therein into the third chamber 1012. That is, an external force may be applied to the second blister chamber to collapse the blister chamber 1008 and move the fluid to the third chamber 1012. Accordingly, the solution is brought into contact with the lateral flow assay strip 1014 via the absorbent pad 1016. As the absorbent pad 1016 was pre-saturated with diluent, the concentration of the diluent may be greater than the sample during an initial period where the lateral flow assay strip is running. In some embodiments, the diagnostic test blister pack 1000 may include a check valve configured to prevent fluid from flowing back to the first blister chamber 1002 from the second blister chamber.

In another version, the sample is processed initially in a sample tube, and then injected into a sample port of the blister pack, where it undergoes amplification (e.g., RPA, LAMP, NEAR, or other isothermal amplification process) and then is added to a lateral flow device to be analyzed. In a further embodiment, the swab is mixed with the sample buffer and a lyophilized lysis mix is added when a frangible seal is broken. The sample is then moved to a lyophilized amplification mix comprising the reagents necessary for RPA, LAMP, or other isothermal amplification techniques. Similarly, a diluent is added to the lyophilized mixture when its frangible seal is broken. The sample, after processing, is then added to a lateral flow device to be analyzed. In some embodiments, the lysis is accomplished by enzymatic and/or detergent lysis mechanisms. In a further embodiment, heat lysis is used. That is, the sample is added to the sample buffer and then heat is applied to lyse the sample. After the sample has been lysed, it is then moved to a lyophilized amplification mix chamber (blister). Similarly, a diluent is added to the lyophilized mixture when its frangible seal is broken. The sample, after processing, is then added to a lateral flow device to be analyzed. In some embodiments, each of the steps is separated by a rotary valve, which controls the flow of the sample into the next chamber (e.g., blister).

A further embodiment of the blister pack configuration comprises a swab in conjunction with a blister pack. A sample is taken using a swab. The swab is added to a tube comprising buffer and incubated for 10 minutes at room temperature. Then, a cap comprising one or more lysis reagents is added to the tube. Adding the cap dispenses the lysis reagents into the buffer and sample. The mixture is then heated at 95 ° C. for three minutes but the invention is not so limited. Other temperatures are envisioned. In some embodiments, the heating is accomplished with any heater described herein (e.g., boiling water, a fixed heat source). The reaction mixture is then allowed to cool for 1 minute, but this time period is not limiting as other time periods are envisioned. The resulting reaction mixture is then injected into a sample port of the blister pack (e.g., using a pipette). The cartridge is then sealed with seal tape and then shaken or otherwise agitated for 10 seconds but this time period is not limiting. The cartridge is heated for 20 minutes but this time period also is not limiting. In some embodiments, the cartridge is placed in a user's clothing pocket (e.g., back pocket of pants, front pocket of pants, front pocket of shirt) to heat the cartridge using the user's body heat. The user then pushes on a first blister to release a one or more amplification reagents (e.g., one or more reagents for LAMP, RPA, NEAR, or other isothermal amplification methods). The user presses on a second blister to release the diluent and turns a valve to permit the mixture to proceed to an absorbent pad and/or lateral flow strip after the appropriate amount of processing. The lateral flow strip may indicate whether one or more target nucleic acid sequences are present in the sample. In some embodiments, the results on the lateral flow strip may be interpreted using a mobile software-based application, downloadable to a smart device, such as that described herein.

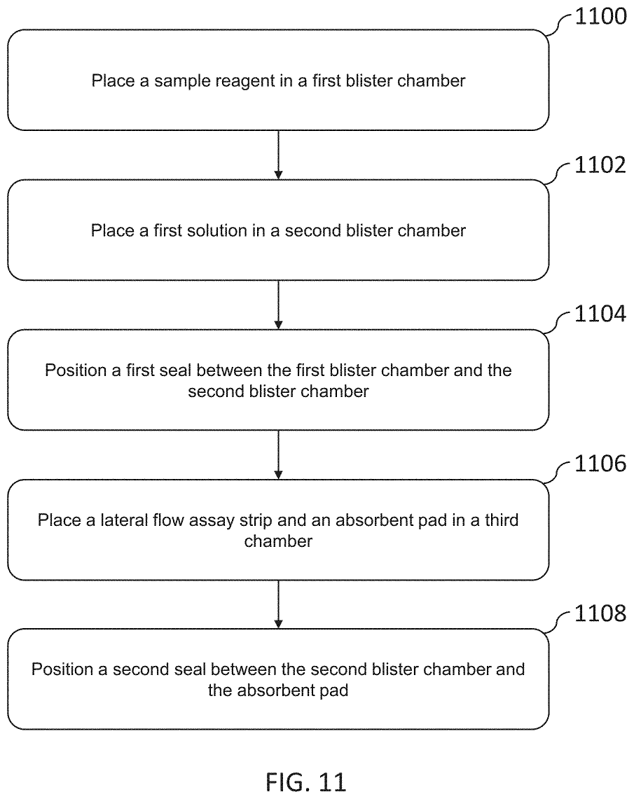

FIG. 11 depicts a flow chart for one embodiment of a method of manufacturing a diagnostic test detection component including one or more blister chambers. In block 1100, a sample reagent is placed in a first blister chamber. In some embodiments, the sample reagent may be a lyophilized solid. In other embodiments, the sample reagent may be a liquid solution. In block 1102, a first solution is placed in a second blister chamber. In some embodiments, the second blister chamber is adjacent to the first blister chamber. The first solution may be a diluent. In step 1104, a first seal may be positioned between the first blister chamber and the second blister chamber. In some embodiments, the seal may be a frangible seal configured to release fluid when opened in an uncontrolled manner. In other embodiments, the seal may be a valve configured to release fluid when opened in a controlled manner. In step 1106, a lateral flow assay strip and absorbent pad are placed in a third chamber. In step 1108, a second seal is positioned between the second blister chamber and the third chamber (e.g., between the second chamber and the lateral flow assay strip). Accordingly, the diagnostic test made by the method of FIG. 4 may include three chambers arranged in sequence. That is, the first chamber may not be directly connected to the third chamber, but rather indirectly through the second chamber.

### Diagnostic Test Applications

The absorbent pad described may be used with any suitable diagnostic test, including the exemplary such test described herein. Diagnostic devices, systems, and methods described herein, including absorbent pads, may be safely and easily operated or conducted by untrained individuals. Unlike prior art diagnostic tests, some embodiments described herein may not require knowledge of even basic laboratory techniques (e.g., pipetting). Similarly, some embodiments described herein may not require expensive laboratory equipment (e.g., thermocyclers). In some embodiments, reagents are contained within a reaction tube, a cartridge, and/or a blister pack, such that users are not exposed to any potentially harmful chemicals.

Diagnostic devices, systems, and methods described herein are also highly sensitive and accurate. In some embodiments, the diagnostic devices, systems, and methods are configured to detect one or more target nucleic acid sequences using nucleic acid amplification (e.g., an isothermal nucleic acid amplification method). Through nucleic acid amplification, the diagnostic devices, systems, and methods are able to accurately detect the presence of extremely small amounts of a target nucleic acid. In certain cases, for example, the diagnostic devices, systems, and methods can detect 1 pM or less, or 10 aM or less.

As a result, the diagnostic devices, systems, and methods described herein may be useful in a wide variety of contexts. For example, in some cases, the diagnostic devices and systems may be available over the counter for use by consumers. In such cases, untrained consumers may be able to self-administer the diagnostic test (or administer the test to friends and family members) in their own homes (or any other location of their choosing). In some cases, the diagnostic devices, systems, or methods may be operated or performed by employees or volunteers of an organization (e.g., a school, a medical office, a business). For example, a school (e.g., an elementary school, a high school, a university) may test its students, teachers, and/or administrators, a medical office (e.g., a doctor's office, a dentist's office) may test its patients, or a business may test its employees for a particular disease. In each case, the diagnostic devices, systems, or methods may be operated or performed by the test subjects (e.g., students, teachers, patients, employees) or by designated individuals (e.g., a school nurse, a teacher, a school administrator, a receptionist).

In some embodiments, diagnostic devices described herein are relatively small. In certain cases, for example, a cartridge is approximately the size of a pen or a marker. Thus, unlike diagnostic tests that require bulky equipment, diagnostic devices and systems described herein may be easily transported and/or easily stored in homes and businesses. In some embodiments, the diagnostic devices and systems are relatively inexpensive. Since no expensive laboratory equipment (e.g., a thermocycler) is required, diagnostic devices, systems, and methods described herein may be more cost effective than known diagnostic tests.

In some embodiments, any reagents contained within a diagnostic device or system described herein may be thermostabilized, and the diagnostic device or system may be shelf stable for a relatively long period of time. In certain embodiments, for example, the diagnostic device or system may be stored at room temperature (e.g., 20° C. to 25° C.) for a relatively long period of time (e.g., at least 1 month, at least 3 months, at least 6 months, at least 9 months, at least 1 year, at least 5 years, at least 10 years). In certain embodiments, the diagnostic device or system may be stored across a range of temperatures (e.g., 0° C. to 20° C., 0° C. to 37° C., 0° C. to 60° C., 0° C. to 90° C., 20° C. to 37° C., 20° C. to 60° C., 20° C. to 90° C., 37° C. to 60° C., 37° C. to 90° C., 60° C. to 90° C.) for a relatively long period of time (e.g., at least 1 month, at least 3 months, at least 6 months, at least 9 months, at least 1 year, at least 5 years, at least 10 years).

### Target Nucleic Acid Sequences

The diagnostic devices, systems, and methods described herein may be used to detect the presence or absence of any target nucleic acid sequence (e.g., from any pathogen of interest) or multiple target nucleic acid sequences. Target nucleic acid sequences may be associated with a variety of diseases or disorders. In some embodiments, the diagnostic devices, systems, and methods are used to diagnose at least one disease or disorder caused by a pathogen. In certain instances, the diagnostic devices, systems, and methods are configured to detect a nucleic acid encoding a protein (e.g., a nucleocapsid protein) of SARS-CoV-2, which is the virus that causes COVID-19. In some embodiments, the diagnostic devices, systems, and methods are used to diagnose at least one disease or disorder caused by a virus, bacteria, fungus, protozoan, parasite, and/or cancer cell. Of course, a diagnostic test according to exemplary embodiments described herein (e.g., a blister pack) may be employed to detect any desired target nucleic acid sequence, as the present disclosure is not so limited.

### Sample Collection

In some embodiments, a diagnostic method comprises collecting a sample from a subject (e.g., a human subject, an animal subject). In some embodiments, a diagnostic system comprises a sample-collecting component configured to collect a sample from a subject (e.g., a human subject, an animal subject). Exemplary samples include bodily fluids (e.g. mucus, saliva, blood, serum, plasma, amniotic fluid, sputum, urine, cerebrospinal fluid, lymph, tear fluid, feces, or gastric fluid), cell scrapings (e.g., a scraping from the mouth or interior cheek), exhaled breath particles, tissue extracts, culture media (e.g., a liquid in which a cell, such as a pathogen cell, has been grown), environmental samples, agricultural products or other foodstuffs, and their extracts. In some embodiments, the sample comprises a nasal secretion. In certain instances, for example, the sample is an anterior nares specimen. An anterior nares specimen may be collected from a subject by inserting a swab element of a sample-collecting component into one or both nostrils of the subject for a period of time. In some embodiments, the sample comprises a cell scraping. In certain embodiments, the cell scraping is collected from the mouth or interior cheek. The cell scraping may be collected using a brush or scraping device formulated for this purpose. The sample may be self-collected by the subject or may be collected by another individual (e.g., a family member, a friend, a coworker, a health care professional) using a sample-collecting component described herein.

### Lysis of Sample

In some embodiments, lysis is performed by chemical lysis (e.g., exposing a sample to one or more lysis reagents) and/or thermal lysis (e.g., heating a sample). Chemical lysis may be performed by one or more lysis reagents. In some embodiments, the one or more lysis reagents comprise one or more enzymes. In some embodiments, the one or more lysis reagents comprise one or more detergents. In some embodiments, cell lysis is accomplished by applying heat to a sample (thermal lysis). In certain instances, thermal lysis is performed by applying a lysis heating protocol comprising heating the sample at one or more temperatures for one or more time periods using any heater described herein. In some embodiments, a lysis heating protocol comprises heating the sample at a first temperature for a first time period.

### Nucleic Acid Amplification

Following lysis, one or more target nucleic acids (e.g., a nucleic acid of a target pathogen) may be amplified. In some cases, a target pathogen has RNA as its genetic material. In certain instances, for example, a target pathogen is an RNA virus (e.g., a coronavirus, an influenza virus). In some such cases, the target pathogen's RNA may need to be reverse transcribed to DNA prior to amplification. In some embodiments, reverse transcription is performed by exposing lysate to one or more reverse transcription reagents. In certain instances, the one or more reverse transcription reagents comprise a reverse transcriptase, a DNA-dependent polymerase, and/or a ribonuclease (RNase). In some embodiments, DNA may be amplified according to any nucleic acid amplification method known in the art.

### LAMP

In some embodiments, the nucleic acid amplification reagents are LAMP reagents. LAMP refers to a method of amplifying a target nucleic acid using at least four primers through the creation of a series of stem-loop structures. Due to its use of multiple primers, LAMP may be highly specific for a target nucleic acid sequence.

### RPA

In some embodiments, the nucleic acid amplification reagents are RPA reagents. RPA generally refers to a method of amplifying a target nucleic acid using a recombinase, a single-stranded DNA binding protein, and a strand-displacing polymerase.

### Nicking Enzyme Amplification Reaction (NEAR)

In some embodiments, amplification of one or more target nucleic acids is accomplished through the use of a nicking enzyme amplification reaction (NEAR) reaction. NEAR generally refers to a method for amplifying a target nucleic acid using a nicking endonuclease and a strand displacing DNA polymerase. In some cases, NEAR may allow for amplification of very small amplicons.

### Molecular Switches

As described herein, a sample undergoes lysis and amplification prior to detection. In certain embodiments, one or more (and, in some cases, all) of the reagents necessary for lysis and/or amplification are present in a single pellet or tablet. In some embodiments, a pellet or tablet may comprise two or more enzymes, and it may be necessary for the enzymes to be activated in a particular order. Therefore, in some embodiments, the enzyme tablet further comprises one or more molecular switches. Molecular switches, as described herein, are molecules that, in response to certain conditions, reversibly switch between two or more stable states. In some embodiments, the condition that causes the molecular switch to change its configuration is pH, light, temperature, an electric current, microenvironment, or the presence of ions and other ligands. In one embodiment, the condition is heat. In some embodiments, the molecular switches described herein are aptamers. Aptamers generally refer to oligonucleotides or peptides that bind to specific target molecules (e.g., the enzymes described herein). The aptamers, upon exposure to heat or other conditions, may dissociate from the enzymes. With the use of molecular switches, the processes described herein (e.g., lysis, decontamination, reverse transcription, and amplification) may be performed in a single test tube with a single enzymatic tablet.

### Detection

In some embodiments, amplified nucleic acids (i.e., amplicons) may be detected using any suitable methods. In some embodiments, one or more target nucleic acid sequences are detected using a lateral flow assay strip.

In some embodiments, the one or more fluid-transporting layers of the lateral flow assay strip comprise a plurality of fibers (e.g., woven or non-woven fabrics). In some embodiments, the one or more fluid-transporting layers comprise a plurality of pores. In some embodiments, pores and/or interstices between fibers may advantageously facilitate fluid transport (e.g., via capillary action). The pores may have any suitable average pore size. In certain embodiments, the plurality of pores has an average pore size of 30 μm or less, 25 μm or less, 20 μm or less, 15 μm or less, 10 μm or less, 5 μm or less, 2 μm or less, 1 μm or less, 0.8 μm or less, 0.6 μm or less, 0.4 μm or less, or 0.2 μm or less. In certain embodiments, the plurality of pores has an average pore size of at least 0.1 μm, at least 0.3 μm, at least 0.5 μm, at least 0.7 μm, at least 0.9 μm, at least 1 μm, at least 2 μm, at least 5 μm, at least 10 μm, at least 15 μm, at least 20 μm, at least 25 μm, or at least 30 μm.

The one or more fluid-transporting layers of the lateral flow assay strip may have any suitable porosity. In some embodiments, the one or more fluid-transporting layers have a porosity of at least 10%, at least 20%, at least 30%, at least 40%, at least 50%, or at least 60%. In some embodiments, the one or more fluid-transporting layers have a porosity in a range from 10% to 20%, 10% to 30%, 10% to 40%, 10% to 50%, 10% to 60%, 20% to 40%, 20% to 50%, 20% to 60%, 30% to 50%, 30% to 60%, 40% to 60%, or 50% to 60%.

In some embodiments, a fluidic sample is introduced to a first sub-region (e.g., a sample pad) of the lateral flow assay strip. In certain embodiments, the fluidic sample subsequently flows through a second sub-region (e.g., a particle conjugate pad) comprising a plurality of labeled particles. In some cases, the particles comprise gold nanoparticles (e.g., colloidal gold nanoparticles). The particles may be labeled with any suitable label. Non-limiting examples of suitable labels include biotin, streptavidin, fluorescein isothiocyanate (FITC), fluorescein amidite (FAM), fluorescein, and digoxigenin (DIG). In some cases, as an amplicon-containing fluidic sample flows through the second sub-region (e.g., a particle conjugate pad), a labeled nanoparticle binds to a label of an amplicon, thereby forming a particle-amplicon conjugate.

In some embodiments, the fluidic sample (e.g., comprising a particle-amplicon conjugate) subsequently flows through a third sub-region (e.g., a test pad) comprising one or more test lines. In some embodiments, a first test line comprises a capture reagent (e.g., an immobilized antibody) configured to detect a first target nucleic acid. In some embodiments, a particle-amplicon conjugate may be captured by one or more capture reagents (e.g., immobilized antibodies), and an opaque marking may appear. The marking may have any suitable shape or pattern (e.g., one or more straight lines, curved lines, dots, squares, check marks, x marks).

In certain embodiments, the lateral flow assay strip comprises one or more additional test lines. In some instances, each test line of the lateral flow assay strip is configured to detect a different target nucleic acid. In some instances, two or more test lines of the lateral flow assay strip are configured to detect the same target nucleic acid. The test line(s) may have any suitable shape or pattern (e.g., one or more straight lines, curved lines, dots, squares, check marks, x marks).

In certain embodiments, the third sub-region (e.g., the test pad) of the lateral flow assay strip further comprises one or more control lines. In certain instances, a first control line is a human (or animal) nucleic acid control line. In some embodiments, for example, the human (or animal) nucleic acid control line is configured to detect a nucleic acid (e.g., RNase P) that is generally present in all humans (or animals). In some cases, the human (or animal) nucleic acid control line becoming detectable indicates that a human (or animal) sample was successfully collected, nucleic acids from the sample were amplified, and the amplicons were transported through the lateral flow assay strip. In certain instances, a first control line is a lateral flow control line. In some cases, the lateral flow control line becoming detectable indicates that a liquid was successfully transported through the lateral flow assay strip. In some embodiments, the lateral flow assay strip comprises two or more control lines. The control line(s) may have any suitable shape or pattern (e.g., one or more straight lines, curved lines, dots, squares, check marks, x marks). In some instances, for example, the lateral flow assay strip comprises a human (or animal) nucleic acid control line and a lateral flow control line.

In certain embodiments, the lateral flow assay strip comprises a fourth sub-region (e.g., a wicking area) to absorb fluid flowing through the lateral flow assay strip. Any excess fluid may flow through the fourth sub-region. As an illustrative example, a fluidic sample comprising an amplicon labeled with biotin and FITC may be introduced into a lateral flow assay strip (e.g., through a sample pad of a lateral flow assay strip). In some embodiments, as the labeled amplicon is transported through the lateral flow assay strip (e.g., through a particle conjugate pad of the lateral flow assay strip), a gold nanoparticle labeled with streptavidin may bind to the biotin label of the amplicon. In some cases, the lateral flow assay strip (e.g., a test pad of the lateral flow assay strip) may comprise a first test line comprising an anti-FITC antibody. In some embodiments, the gold nanoparticle-amplicon conjugate may be captured by the anti-FITC antibody, and an opaque band may develop as additional gold nanoparticle-amplicon conjugates are captured by the anti-FITC antibodies of the first test line. In some cases, the lateral flow assay strip (e.g., a test pad of the lateral flow assay strip) further comprises a first lateral flow control line comprising biotin. In some embodiments, excess gold nanoparticles labeled with streptavidin (i.e., gold nanoparticles that were not conjugated to an amplicon) transported through the lateral flow assay strip may bind to the biotin of the first lateral flow control line, demonstrating that liquid was successfully transported to the first lateral flow control line. In certain embodiments, for example, a fluidic sample is exposed to a reagent that undergoes a color change when bound to a target nucleic acid (e.g., viral DNA or RNA), such as with an enzyme-linked immunoassay. In some embodiments, the assay further comprises a stop reagent, such as sulfonic acid. That is, when the fluidic sample is mixed with the reagents, the solution turns a specific color (e.g., red) if the target nucleic acid is present, and the sample is positive. If the solution turns a different color (e.g., green), the target nucleic acid is not present, and the sample is negative.

In some embodiments, the diagnostic device comprises a plurality of lateral flow assay strips. In certain cases, the plurality of lateral flow assay strips may be connected such that a fluidic sample may flow from a first end to a second end of a first lateral flow assay strip (e.g., via capillary action) and may then flow from the second end of the first lateral flow assay trip to a first end of a second lateral flow assay strip. In certain instances, the diagnostic device comprises a series of lateral flow strips that snap or lock together. In some cases, the diagnostic device comprises one or more lateral flow assay strips that have been impregnated with one or more reagents (e.g., lysis reagents, nucleic acid amplification reagents, CRISPR/Cas detection reagents). In certain embodiments, the one or more reagents may be in solid form (e.g., lyophilized, dried, crystallized, air jetted), and one or more buffers may be added to activate the solid reagents and move the sample to the next strip. In some embodiments, the strips have dams or gaps to impede fluid flow to give a reaction (e.g., lysis, amplification) sufficient time to occur.

### Instructions & Software

In some embodiments, a diagnostic system comprises instructions for using a diagnostic device and/or otherwise performing a diagnostic test method. The instructions may include instructions for the use, assembly, and/or storage of the diagnostic device and any other components associated with the diagnostic system. The instructions may be provided in any form recognizable by one of ordinary skill in the art as a suitable vehicle for containing such instructions. For example, the instructions may be written or published, verbal, audible (e.g., telephonic), digital, optical, visual (e.g., videotape, DVD, etc.) or electronic communications (including Internet or web-based communications). In some embodiments, the instructions are provided as part of a software-based application. In certain cases, the application can be downloaded to a smartphone or device, and then guides a user through steps to use the diagnostic device.

In some embodiments, a software-based application may be connected (e.g., via a wired or wireless connection) to one or more components of a diagnostic system. In certain embodiments, for example, a heater may be controlled by a software-based application. In some cases, a user may select an appropriate heating protocol through the software-based application. In some cases, an appropriate heating protocol may be selected remotely (e.g., not by the immediate user). In some cases, the software-based application may store information (e.g., regarding temperatures used during the processing steps) from the heater.

In some embodiments, a diagnostic system comprises or is associated with software to read and/or analyze test results. In some embodiments, a device (e.g., a camera, a smartphone) is used to generate an image of a test result (e.g., one or more lines detectable on a lateral flow assay strip). In some embodiments, a user may use an electronic device (e.g., a smartphone, a tablet, a camera) to acquire an image of the visible portion of the lateral flow assay strip. In some embodiments, software running on the electronic device may be used to analyze the image (e.g., by comparing any lines or other markings that appear on the lateral flow assay strip with known patterns of markings). That result may be communicated directly to a user or to a medical professional. In some cases, the test result may be further communicated to a remote database server. In some embodiments, the remote database server stores test results as well as user information such as at least one of name, social security number, date of birth, address, phone number, email address, medical history, and medications.

While the present teachings have been described in conjunction with various embodiments and examples, it is not intended that the present teachings be limited to such embodiments or examples. On the contrary, the present teachings encompass various alternatives, modifications, and equivalents, as will be appreciated by those of skill in the art. Accordingly, the foregoing description and drawings are by way of example only.

What is claimed is:

1. A component of a diagnostic test comprising:
an absorbent pad at least partially saturated with a first solution, wherein the absorbent pad is fluidly coupled between a reservoir that receives a sample and a readout element, wherein the sample flows between the reservoir and the readout element through the absorbent pad.

2. The component of claim 1, wherein the first solution is a diluent.

3. The component of claim 1, wherein a saturation of the absorbent pad is less than a threshold saturation for running the readout element.

4. The component of claim 3, wherein the absorbent pad has an average pore size configured to prevent liquid from flowing to the readout element until the threshold saturation is reached.

5. The component of claim 1, wherein the sample and the first solution are configured to mix in the absorbent pad.

6. The component of claim 5, wherein the absorbent pad is configured to deliver a mixture of the first solution and the sample to the readout element, wherein a concentration of the first solution in the mixture is greater than a concentration of the sample.

7. The component of claim 1, wherein the readout element comprises a lateral flow assay strip.

8. A diagnostic test kit comprising:
a lateral flow assay strip;
an absorbent pad fluidly connected to the lateral flow assay strip, wherein the absorbent pad is at least partially saturated with a first solution;
a fluidic channel fluidly connected to the absorbent pad; and
a receptacle fluidly connected to the fluidic channel and configured to receive and fluidly connect to a first reservoir containing a sample, wherein fluidly connecting the first reservoir to the receptacle allows the sample to flow from the first reservoir to the absorbent pad via the fluidic channel.

9. The diagnostic test kit of claim 8, further comprising a seal positioned between the absorbent pad and the receptacle, wherein fluidly connecting the first reservoir to the receptacle opens the seal.

10. The diagnostic test kit of claim 8, wherein the receptacle includes a needle configured to puncture the first reservoir when the first reservoir is moved against the needle.

11. The diagnostic test kit of claim 8, wherein the receptacle includes a blade configured to puncture the first reservoir when the first reservoir is moved against the blade.

12. The diagnostic test kit of claim 8, wherein the first solution is a diluent.

13. The diagnostic test kit of claim 8, wherein a saturation of the absorbent pad is less than a threshold saturation for running the lateral flow assay strip.

14. The diagnostic test kit of claim 13, wherein the absorbent pad has an average pore size configured to prevent liquid from flowing to the lateral flow assay strip until the threshold saturation is reached.

15. The diagnostic test kit of claim 8, wherein the sample and the first solution are configured to mix in the absorbent pad.

16. The diagnostic test kit of claim 15, wherein the absorbent pad is configured to deliver a mixture of the first solution and the sample to the lateral flow assay strip, wherein a concentration of the first solution in the mixture is greater than a concentration of the sample.

17. A method of manufacturing a diagnostic test, comprising:
placing a readout element and an absorbent pad in a housing such that the readout element is in fluid communication with the absorbent pad;
at least partially filling the absorbent pad with a first solution; and
providing a vial for receiving a sample from a patient such that the vial may fluidly connect to the housing, thereby enabling the sample to flow to the absorbent pad.

18. The method of claim 17, wherein the housing includes a receptacle configured to receive the vial.

19. The method of claim 18, wherein at least partially filling the absorbent pad includes dispensing the first solution into the receptacle.

20. The method of claim 19, wherein at least partially filling the absorbent pad includes saturating the absorbent pad to a saturation less than a threshold saturation for running the readout element.

21. The method of claim 20, wherein the absorbent pad has an average pore size configured to prevent liquid from flowing to the readout element until the threshold saturation is reached.

22. The method of claim 17, further comprising placing a seal in the housing to seal the first solution in the absorbent pad.

23. The method of claim 17, wherein the first solution is a diluent.

24. The method of claim 17, wherein the readout element comprises a lateral flow assay strip.
